SEQUENCE LISTING (18 sequences)

SEQ ID NO: 1 - DNA, 1275 bp, Homo sapiens
atgagcgacagtaaatgtgacagtcagttttatagtgtgcaagtggcagactcaaccttcactgtcctaaaacgttaccagcagctgaaaccaattggctctggggcccaagggattgtttgtgctgcatttgatacagttcttgggataaatgttgcagtcaagaaactaagccgtccttttcagaaccaaactcatgcaaagagagcttatcgtgaacttgtcctcttaaaatgtgtcaatcataaaaatataattagtttgttaaatgtgtttacaccacaaaaaactctagaagaatttcaagatgtgtatttggttatggaattaatggatgctaacttatgtcaggttattcacatggagctggatcatgaaagaatgtcctaccttctttaccagatgctttgtggtattaaacatctgcattcagctggtataattcatagagatttgaagcctagcaacattgttgtgaaatcagactgcaccctgaagatccttgactttggcctggcccggacagcgtgcactaacttcatgatgaccccttacgtggtgacacggtactaccgggcgcccgaagtcatcctgggtatgggctacaaagagaacgttgatatctggtcagtgggttgcatcatgggagagctggtgaaaggttgtgtgatattccaaggcactgaccatattgatcagtggaataaagttattgagcagctgggaacaccatcagcagagttcatgaagaaacttcagccaactgtgaggaattatgtcgaaaacagaccaaagtatcctggaatcaaatttgaagaactctttccagattggatattcccatcagaatctgagcgagacaaaataaaaacaagtcaagccagagatctgttatcaaaaatgttagtgattgatcctgacaagcggatctctgtagacgaagctctgcgtcacccatacatcactgtttggtatgaccccgccgaagcagaagccccaccacctcaaatttatgatgcccagttggaagaaagagaacatgcaattgaagaatggaaagagctaatttacaaagaagtcatggattgggaagaaagaagcaagaatggtgttgtaaaagatcagccttcagatgcagcagtaagtagcaacgccactccttctcagtcttcatcgatcaatgacatttcatccatgtccactgagcagacgctggcctcagacacagacagcagtcttgatgcctcgacgggaccccttgaaggctgtcgatga

SEQ ID NO: 2 - DNA, 1149 bp, Homo sapiens
atgagcgacagtaaatgtgacagtcagttttatagtgtgcaagtggcagactcaaccttcactgtcctaaaacgttaccagcagctgaaaccaattggctctggggcccaagggattgtttgtgctgcatttgatacagttcttgggataaatgttgcagtcaagaaactaagccgtccttttcagaaccaaactcatgcaaagagagcttatcgtgaacttgtcctcttaaaatgtgtcaatcataaaaatataattagtttgttaaatgtgtttacaccacaaaaaactctagaagaatttcaagatgtgtatttggttatggaattaatggatgctaacttatgtcaggttattcacatggagctggatcatgaaagaatgtcctaccttctttaccagatgctttgtggtattaaacatctgcattcagctggtataattcatagagatttgaagcctagcaacattgttgtgaaatcagactgcaccctgaagatccttgactttggcctggcccggacagcgtgcactaacttcatgatgaccccttacgtggtgacacggtactaccgggcgcccgaagtcatcctgggtatgggctacaaagagaacgttgatatctggtcagtgggttgcatcatgggagagctggtgaaaggttgtgtgatattccaaggcactgaccatattgatcagtggaataaagttattgagcagctgggaacaccatcagcagagttcatgaagaaacttcagccaactgtgaggaattatgtcgaaaacagaccaaagtatcctggaatcaaatttgaagaactctttccagattggatattcccatcagaatctgagcgagacaaaataaaaacaagtcaagccagagatctgttatcaaaaatgttagtgattgatcctgacaagcggatctctgtagacgaagctctgcgtcacccatacatcactgtttggtatgaccccgccgaagcagaagccccaccacctcaaatttatgatgcccagttggaagaaagagaacatgcaattgaagaatggaaagagctaatttacaaagaagtcatggattgggaagaaagaagcaagaatggtgttgtaaaagatcagccttcagcacagatgcagcagtaa

SEQ ID NO: 3 - DNA, 1275 bp, Homo sapiens
atgagcgacagtaaatgtgacagtcagttttatagtgtgcaagtggcagactcaaccttcactgtcctaaaacgttaccagcagctgaaaccaattggctctggggcccaagggattgtttgtgctgcatttgatacagttcttgggataaatgttgcagtcaagaaactaagccgtccttttcagaaccaaactcatgcaaagagagcttatcgtgaacttgtcctcttaaaatgtgtcaatcataaaaatataattagtttgttaaatgtgtttacaccacaaaaaactctagaagaatttcaagatgtgtatttggttatggaattaatggatgctaacttatgtcaggttattcacatggagcgagctggtgaaaggttgtgctaccttctttaccagatgctttgtggtattaaacatctgcattcagctggtataattcatagagatttgaagcctagcaacattgttgtgaaatcagactgcaccctgaagatccttgactttggcctggcccggacagcgtgcactaacttcatgatgaccccttacgtggtgacacggtactaccgggcgcccgaagtcatcctgggtatgggctacaaagagaacgttgatatctggtcagtcgggtgcatcatggcagaaatggtcctccataaagtcctgttcccgggaagagactatattgatcagtggaataaagttattgagcagctgggaacaccatcagcagagttcatgaagaaacttcagccaactgtgaggaattatgtcgaaaacagaccaaagtatcctggaatcaaatttgaagaactctttccagattggatattcccatcagaatctgagcgagacaaaataaaaacaagtcaagccagagatctgttatcaaaaatgttagtgattgatcctgacaagcggatctctgtagacgaagctctgcgtcacccatacatcactgtttggtatgaccccgccgaagcagaagccccaccacctcaaatttatgatgcccagttggaagaaagagaacatgcaattgaagaatggaaagagctaatttacaaagaagtcatggattgggaagaaagaagcaagaatggtgttgtaaaagatcagccttcagatgcagcagtaagtagcaacgccactccttctcagtcttcatcgatcaatgacatttcatccatgtccactgagcagacgctggcctcagacacagacagcagtcttgatgcctcgacgggaccccttgaaggctgtcgatga

SEQ ID NO: 4 - protein, 424 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMGELVKGCVIFQGTDHIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPSDAAVSSNATPSQSSSINDISSMSTEQTLASDTDSSLDASTGPLEGCR

SEQ ID NO: 5 - protein, 382 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMELDHERMSYLLYQMLCGIKHLHSAGIIHRDLKPSNIVVKSDCTLKILDFGLARTACTNFMMTPYVVTRYYRAPEVILGMGYKENVDIWSVGCIMGELVKGCVIFQGTDHIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPSAQMQQ

SEQ ID NO: 6 - protein, 425 aa, Homo sapiens
MSDSKCDSQFYSVQVADSTFTVLKRYQQLKPIGSGAQGIVCAAFDTVLGINVAVKKLSRPFQNQTHAKRAYRELVLLKCVNHKNIISLLNVFTPQKTLEEFQDVYLVMELMDANLCQVIHMERAGERLCYLLYQMLCGIKHLHSAGIIHRDLKPSNIVVKSDCTLKILDFGLARTACTNFMMTPYVVTRYYRAPEVILGMGYKENVDIWSVGCIMAEMVLHKVLFPGRDYIDQWNKVIEQLGTPSAEFMKKLQPTVRNYVENRPKYPGIKFEELFPDWIFPSESERDKIKTSQARDLLSKMLVIDPDKRISVDEALRHPYITVWYDPAEAEAPPPQIYDAQLEEREHAIEEWKELIYKEVMDWEERSKNGVVKDQPSDAAVSSNATPSQSSSINDISSMSTEQTLASDTDSSLDASTGPLEGCRS

SEQ ID NO: 7 - DNA, 2244 bp, Homo sapiens
atggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatag

SEQ ID NO: 8 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 9 - DNA, 1182 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 10 - protein, 393 aa, Homo sapiens [sequence omitted]

SEQ ID NO: 11 - DNA, 981 bp, Homo sapiens
atgtctaatggttatgaagaccacatggccgaagactgcaggggtgacatcgggagaacgaatttgatcgtcaactacctccctcagaacatgacccaggatgagttacgaagcctgttcagcagcattggtgaagttgaatctgcaaaacttattcgggataaagtagcaggacacagcttgggctacggctttgtgaactacgtgaccgcgaaggatgcagagagagcgatcaacacgctgaacggcttgaggctccagtcaaaaaccattaaggtgtcgtatgctcgcccgagctcagaggtgatcaaagacgccaacttgtacatcagcgggctcccgcggaccatgacccagaaggacgtagaagacatgttctctcggtttgggcggatcatcaactcgcgggtcctcgtggatcagactacaggtttgtccagaggggttgcgtttatccggtttgacaaacggtcggaggcagaagaggcaattaccagtttcaatggtcataaacccccaggttcctctgagcccatcgcagtgaagtttgcagccaaccccaaccagaacaaaaacgtggcactcctctcgcagctgtaccactcgccagcgcgacggttcggaggccccgttcaccaccaggcgcagagattcaggttctcccccatgggcgtcgatcacatgagcgggctctctggcgtcaacgtgccaggaaacgcctcctccggctggtgcattttcatctacaacctggggcaggatgccgacgaggggatcctctggcagatgtttgggccgtttggtgccgtcaccaatgtgaaagtgatccgcgacttcaacaccaacaagtgcaaagggtttggctttgtgaccatgacaaactatgaagaagccgcgatggccatagccagcctgaacggctaccgcctgggggacaaaatcttacaggtttccttcaaaaccaacaagtcccacaaataa

SEQ ID NO: 12 - protein, 326 aa, Homo sapiens
MSNGYEDHMAEDCRGDIGRTNLIVNYLPQNMTQDELRSLFSSIGEVESAKLIRDKVAGHSLGYGFVNYVTAKDAERAINTLNGLRLQSKTIKVSYARPSSEVIKDANLYISGLPRTMTQKDVEDMFSRFGRIINSRVLVDQTTGLSRGVAFIRFDKRSEAEEAITSFNGHKPPGSSEPIAVKFAANPNQNKNVALLSQLYHSPARRFGGPVHHQAQRFRFSPMGVDHMSGLSGVNVPGNASSGWCIFIYNLGQDADEGILWQMFGPFGAVTNVKVIRDFNTNKCKGFGFVTMTNYEEAAMAIASLNGYRLGDKILQVSFKTNKSHK

SEQ ID NO: 13 - DNA, 21 bp, Artificial Sequence
  Made in a lab
ctaattccaagttccataccc

SEQ ID NO: 14 - DNA, 21 bp, Artificial Sequence
  Made in a lab
ctgaagaatctggtggtgaag

SEQ ID NO: 15 - DNA, 22 bp, Artificial Sequence
  Made in a lab
ccaggaagggactatattgatc

SEQ ID NO: 16 - DNA, 22 bp, Artificial Sequence
  Made in a lab
tctctcctccaagtccataact

SEQ ID NO: 17 - DNA, 20 bp, Artificial Sequence
  Made in a lab
gaagcctagcaacattgttg

SEQ ID NO: 18 - DNA, 20 bp, Artificial Sequence
  Made in a lab
gatcaatatggtcagtgcct

(19) United States
(12) Patent Application Publication
(10) Pub. No.: US 2011/0060028 A1
(43) Pub. Date: Mar. 10, 2011

(54) COMBINATION THERAPY

(75) Inventor: Josephine Anne Milner, York (GB)

(73) Assignee: UNIVERSITY OF YORK, York (GB)

(21) Appl. No.: 12/673,813

(22) PCT Filed: Aug. 15, 2008
(86) PCT No.: PCT/GB08/02775

§ 371 Date: Nov. 16, 2010

(30) Foreign Application Priority Data
Aug. 17, 2007 (GB) 0716072.4

(51) Int. Cl. A61K 31/713; A61P 35/00

(52) U.S. Cl. 514/44 A

(57) ABSTRACT

This disclosure relates to a SIRT1 polypeptide and a treatment regime that inhibits the activity of SIRT1 and including a method of diagnosis.

The invention relates to a treatment regime suitable to control the activity of a sirtuin, for example SIRT1, typically in the treatment of hyperproliferative diseases such as cancer, and including diagnostic tests that detects differential phosphorylation of sirtuin polypeptides.

Apoptosis is a process by which multi-cellular organisms regulate cell number and differentiation. The process is regulated by factors which either induce or prevent apoptosis. Tumour suppressor proteins have pro-apoptotic activities. Tumour suppressor genes encode proteins which function to inhibit cell growth or division and are therefore important with respect to maintaining proliferation, growth and differentiation of normal cells. Mutations in tumour suppressor genes result In abnormal cell-cycle progression whereby the normal cell-cycle check points which arrest the cell-cycle, when, for example, DNA is damaged, are ignored and damaged cells divide uncontrollably. The products of tumour suppressor genes function in all parts of the cell (e.g. cell surface, cytoplasm, mitochondrion and nucleus) and prevent the passage of damaged cells through the cell-cyde.

Arguably the tumour suppressor gene which has been the subject of the most intense research is p53. p53 encodes a protein which functions as a transcription factor and is a key regulator of the cell division cycle. It was discovered in 1978 as a protein shown to bind with affinity to the SV40 large T antigen. The p53 gene encodes a 393 amino acid polypeptide with a molecular weight of 53kDa. Genes regulated by the transcriptional activity of p53 contain a p53 recognition sequence in their 5′ regions. These genes are activated when the cellular levels of p53 are elevated due to, for example, DNA damage. Examples of genes that respond to p53 include, mdm2, Bax and PIG-3. Bax and PIG-3 are involved in one of the most important functions of p53, the induction of apoptosis.

The ability of mammalian cells to withstand metabolic and genotoxic stress involves SIRT1, a class III histone de-acetylase able to regulate gene expression at several levels. Thus deacetylation of linker histone H1 by SIRT1 enables heterochromatin formation and associated gene silencing¹⁶. SIRT1 also deacetylates core histone H3 and recruitment of SIRT1 to specific promoters results in selective gene silencing^{17,18}. In addition SIRT1 targets several non-histone transcription regulators including the tumour suppressor p53^{19, 20}. De-acetylation of p53 by SIRT1 down-regulates the pro-apoptotic p53 stress response¹¹. SIRT1 and p53 thus counterbalance the cellular response to stress. This balance is dependent upon cellular levels of SIRT1 and p53 since over-expression favours cell survival or apoptosis respectively. Expression levels of p53 and SIRT1 therefore require stringent control. For p53 this is largely achieved through regulation of p53 protein stability^{21, 22}. For SIRT1 a transcriptional feed-back mechanism operates in which SIRT1 forms a complex with the transcription repressor hypermethylated in cancer 1 (HIC1) and selectively suppresses transcription from the SIRT1 promoter^{12, 13}. SIRT1 expression is also regulated at the level of mRNA stability via the RNA-binding protein HuR which binds and stabilises SIRT1 mRNA^{14, 15}.

In a large part the control of cell-cycle progression in eukaryotic cells is controlled by phosphorylation and de-phosphorylation of key regulators by kinases and phophatases respectively.

In mammals there are three families of Mitogen Activated Protein Kinase (MAPK). One family member is the c-jun NH₂-terminal kinases (JNK). In humans there are three JNK kinases encoded by three genes; JNK1, JNK2 and JNK3. The .JNK kinases exist in separate isoforms which are created by alternative splicing from mRNA. JNK1 produces four isoforms; JNK2 also produces four isoforms; and JNK3 produces 2 isoforms. The different JNK isoforms differ in the protein substrates that they modify. Inhibitors of JNK are well known in the art. For example, WO2005/074921, which is incorporated by reference in its entirety, describes a pyrazolanthone Inhibitor of JNK2 and its use in the treatment of atherosclerosis. Moreover there are commercially available JNK Inhibitors . specific to each JNK kinase; for example see Calbiochem kinase inhibitors at http://www.emdblosciences.com/html/CBC/home.html; in particular for JNK2 specific inhibitors see Bennett et al 2001 Proc Natl Acad Sci USA 98, p13681; Han et al 2001, J Clin. Invest 108, p73; Shin et al 2002 Biochim Biophys. Acta, 1589, p311; which are incorporated by reference in their entirety.

Chemotherapeutic agents are generally more effective at killing cancer cells than normal cells. Examples of these agents are well known in the art, some of which induce apoptosis. For example, etoposide and camptothecin are inhibitors of topoisomerases. As a consequence, DNA replication or DNA repair processes are blocked. Doxorubicin and daunorubicin are DNA intercalators. Doxorubicin has been reported to induce CD95

(Fas/Apo-1) gene expression in a p53-dependent mechanism in human primary endothelial cells. Moreover it has been shown to trigger apoptosis in various cell lines and its application in cancer treatment has revealed that p53 accumulates in cells exposed to doxorubicin.

A further example of a chemotherapeutic agent is 5′Fluorouracil (5-FU). 5-FU is an anti-metabolite drug widely used in treatment of colorectal cancer. 5-FU exerts its anticancer effects through inhibition of thymidylate synthase and incorporation of its metabolites into mRNA and DNA resulting in the blockage of their synthesis. Studies on 5FU have shown a clear role for p53 in cell culture, where the loss of p53 function reduces cellular sensitivity to 5-FU, and in vivo, where a number of clinical' studies have found that mutant p53 over expression correlates with resistance to 5-FU. Typically, when 5-FU is administered to a patient, leucovorin is also administered since it enhances the activity of agents such as 5-FU.

The treatment of cancers using chemotherapy can be an effective means to control disease. However, most chemotherapeutic treatments have undesirable side effects that cause the subject pain and suffering. It is therefore desirable to discover alternative treatment regimes that are more effective and result in a reduction In side effects either by shortening the treatment period or by using lower doses of chemotherapeutic agent.

This disclosure relates to evidence that SIRT1 is regulated by post-translational modification. We show that SIRT1 is variably phosphorylated at serine 27 (S27) and that S27P correlates with elevated SIRT1 protein levels in human cancer cells versus non-cancer cells, despite similar SIRT1 mRNA levels. We also disclose the synergistic effect of combining an inhibitor of JNK2 with a chemotherapeutic agent such as 5-FU.

According to an aspect of the invention there is provided a combined therapeutic composition comprising: an inhibitor of JNK2 and at least one chemotherapeutic agent.

In a preferred embodiment of the Invention JNK2 is encoded by a nucleic acid molecule comprising a nucleic acid sequence as shown in FIGS. 5a, 5b or 5c or a nucleic acid molecule that hybridizes under stringent hybridization conditions to the sequence shown FIGS. 5a, 5b or 5c and that encodes a polypeptide which has the activity of JNK2.

In a preferred embodiment of the invention JNK2 is encoded by a nucleic acid molecule as represented in FIG. 5a, 5b or 5c.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Strinqency (allows sequences that share at least 90% identity to hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (allows sequences that share at least 80% Identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (allows sequences that share at least 50% identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

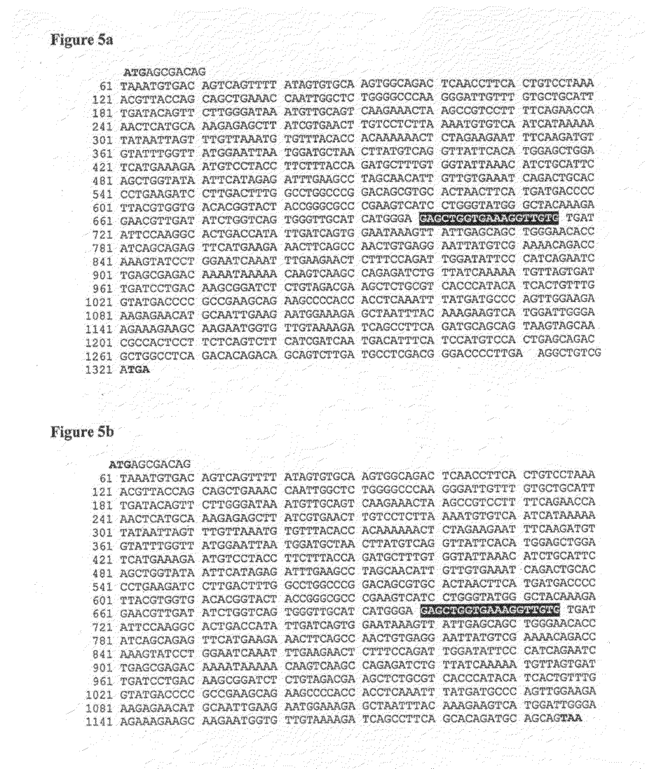

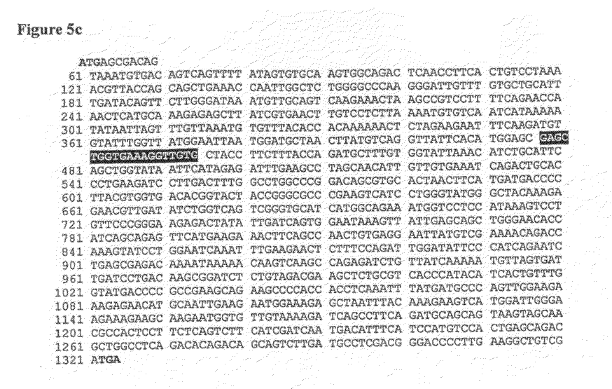

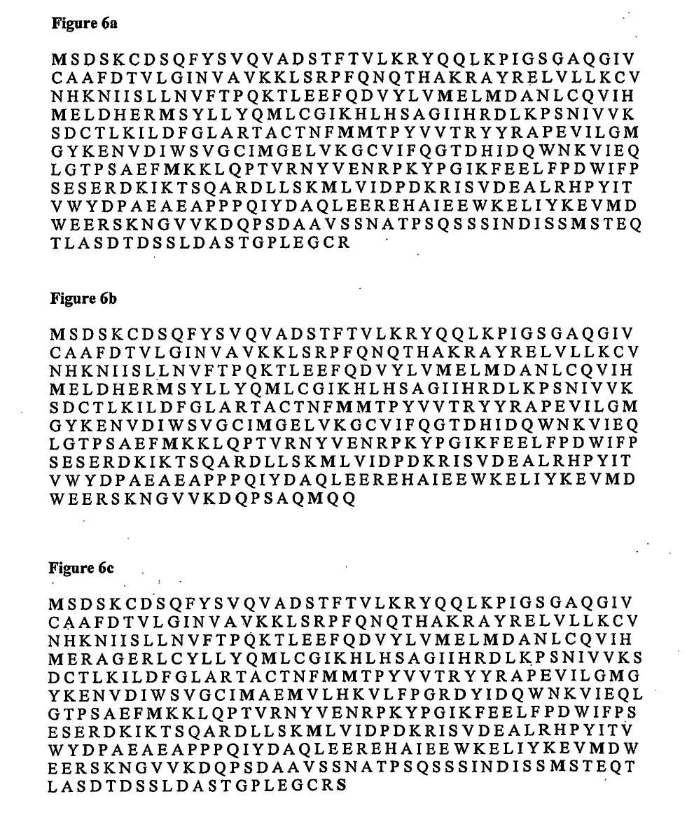

In a preferred embodiment of the invention said nucleic acid molecule encodes a polypeptide as represented by the amino acid sequence as shown in FIGS. 6a, 6b or 6c.

In a preferred embodiment of the invention said inhibitor of JNK2 is a siRNA molecule derived from the nucleic acid sequence in FIG. 5a, 5b or 5c.

A technique to specifically ablate gene function is through the introduction of double stranded RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded

RNA molecule. The siRNA molecule is typically derived from exons of the gene which is to be ablated. The mechanism of RNA interference is being elucidated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA. In our co-pending application WO2006125977 we disclose a modified siRNA-DNA construct (termed ‘crook’ siRNA). When transfected into mammalian cells crook siRNA Induces selective mRNA knock-down equivalent to its unmodified siRNA counterpart. This bi-functional siRNA and the content of WO2006125977 are incorporated by reference in its entirety.

In a preferred embodiment of the invention said JNK2 siRNA is a hybrid nucleic acid molecule comprising a first part that comprises a duplex ribonucleic acid (RNA) molecule and a second part that comprises a single stranded deoxyribonucleic acid (DNA) molecule.

In a preferred embodiment of the invention said single stranded DNA molecule is contiguous with the sense strand of said duplex RNA molecule.

In an alternative preferred embodiment of the invention said single stranded DNA molecule is contiguous with the antisense strand of said duplex RNA molecule.

In a preferred embodiment of the invention said single stranded DNA molecule is extended and is contiguous with both sense and antisense strands of said duplex RNA molecule.

In a preferred embodiment of the invention said single stranded DNA molecule comprises a 3′ terminal nucleic acid sequence wherein said sequence is adapted over at least part of its length to anneal by complementary base pairing to a part of said single stranded DNA to form a double stranded DNA structure.

In a preferred embodiment of the invention said single stranded DNA molecule comprises at least one copy of the sequence d (GCGAAGC).

Typically the single stranded DNA molecule is at least 7 nucleotides in length. Preferably said single stranded DNA molecule is between 10-40 nucleotide bases in length, more preferably 15-30 nucleotides in length.

In a preferred embodiment of the invention said duplex RNA molecule is at least 18 base pairs in length.

In a further preferred embodiment of the invention said duplex RNA molecule is between 19 bp and 1000 bp in length. More preferably the length of said duplex RNA molecule is at least 30 bp; at least 40 bp; at least 50 bp; at least 60 bp; at least 70 bp; at least 80 bp; or at least 90 bp.

In a yet further preferred embodiment of the invention said duplex RNA molecule is at least 100 bp; at least 200 bp; at least 300 bp; at least 400 bp; at least 500 bp; at least at least 600 bp; at least 700 bp; at least 800 bp; at least 900 bp; or at least 1000 bp in length.

Preferably said duplex RNA molecule is between 18 bp and 29 bp in length. More preferably still said duplex RNA molecule is between 21 bp and 27 bp in length. Preferably said duplex RNA molecule is about 21 bp in length.

In an alternative preferred embodiment of the invention said inhibitor is a polypeptide or peptide; preferably a modified peptide inhibitor.

In a preferred method of the invention said agent Is a peptide or polypeptide.

In a preferred method of the invention said peptide is at least 6 amino acid residues in length. Preferably the length of said peptide/polypeptide is selected from the group consisting of: at least 7 amino acid residues; 8, 9, 10, 11, 12, 13, 14, 15, 16, 17, 18, 19, or 20 amino acid residues in length. Alternatively the length of said peptide/polypeptide is at least 20 amino acid residues; 30; 40; 50; 60; 70; 80; 90; or 100 amino acid residues in length.

It will be apparent to one skilled in the art that modification to the amino acid sequence of peptide agents could enhance the binding and/or stability of the peptide with respect to its target sequence. In addition, modification of the peptide may also increase the in vivo stability of the peptide thereby reducing the effective amount of peptide necessary to inhibit JNK2. This would advantageously reduce undesirable side effects which may result in vivo. Modifications include, by example, acetylation and amidation. Alternatively or preferably, said modification includes the use of modified amino acids in the production of recombinant or synthetic forms of peptides. It will be apparent to one skilled in the art that modified amino acids include, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, omithine. Other modifications include amino acids with a C₂, C₃ or C₄ alkyl R group optionally substituted by 1, 2 or 3 substituents selected from halo (eg F, Br, I), hydroxy or C₁-C₄ alkoxy. Modifications also include, by example and not by way of limitation, acetylation and amidation of amino and carboxy-terminal amino acids.

It will also be apparent to one skilled in the art that peptides could be modified by cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

In a preferred embodiment of the invention said polypeptide is an antibody; preferably a monoclonal antibody or fragment thereof, that binds JNK2.

Various fragments antibodies are known in the art, (i.e., Fab, Fab₂, F(ab′)₂, Fv, Fc, Fd, scFvs). A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact antibody molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen.

A fragment could also be a single chain polypeptide containing only one light chain variable region, or a fragment thereof that contains the three Complementarity

Determining Region (CDRs) of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No 6,248,516. Fv fragments or single region (domain) fragments are typically generated by expression In host cell lines of the relevant Identified regions. These and other immunoglobulin or antibody fragments are within the scope of the invention and are described in standard immunology textbooks. Molecular biology now allows direct synthesis (via expression in cells or chemically) of these fragments, as well as synthesis of combinations thereof: A fragment of an antibody or immunoglobulin can also have bispecific function.

In a preferred embodiment of the invention said chemotherapuetic agent is an anti-metabolic drug.

In a preferred embodiment of the invention said drug is a purine analogue. In an alternative preferred embodiment of the invention said drug is a pyrimidine analogue.

Purine analogues are known in the art; for example thioguanine is used to treat acute leukaemia; fludarabine inhibits the function of DNA polymerases, DNA primases and DNA ligases and is specific for cell-cycle S-phase; pentostatin and cladribine are adenosine analogues and are effective against hairy cell leukaemias. Pyrimidine analogues are similarly known in the art. For example, 5-fluorouracil (5-FU), floxuridine and cytosine arabinoside. 5-FU has been used for many years in the treatment of breast, colorectal cancer, pancreatic and other cancers. 5-FU can also been formed from the pro-drug capecitabine which is converted to 5-FU in the tumour.

In a preferred embodiment of the invention said chemotherapeutic agent is 5-fluorouracil.

In a preferred embodiment of the invention said anti-metabolic drug is administered with leucovorin.

Leucovorin, also known as folinic acid, is administered as an adjuvant in cancer chemotherapy and which enhances the inhibitory effects of 5-FU on thymidylate synthase.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an inhibitor/agent according to the invention for producing the desired response in a unit of weight or volume suitable for administration to a patient. The response can, for example, be measured by determining regression of a tumour, decrease of disease symptoms, modulation of apoptosis, etc.

The doses of the inhibitor/agent according to the invention administered to a subject can be chosen in accordance with different parameters, in particular in accordance with the mode of administration used and the state of the subject. Other factors include the desired period of treatment. In the event that a response in a subject is insufficient at the Initial doses applied, higher doses (or effectively higher doses by a different, more localized delivery route) may be employed to the extent that patient tolerance permits.

Other protocols for the administration of compositions will be known to one of ordinary skill in the art, in which the dose amount, schedule of injections, sites of injections, mode of administration (e.g., intra-tumour) and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” in this context denotes an organic or inorganic ingredient, natural or synthetic, with which the active ingredient is combined to facilitate the application, e.g. liposome or immuno-liposome. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion or as a gel. Compositions may be administered as aerosols and inhaled.

Compositions suitable for parenteral administration conveniently comprise a sterile aqueous or non-aqueous preparation of nucleic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the Invention there is provided a method to diagnosis a disease condition that is associated with the phosphorylation of a sirtuin polypeptide comprising:
i) providing an isolated sample comprising a cell to be tested;
ii) determining the phosphorylation of at least one amino acid residue in a sirtuin polypeptide; and
iii) comparing the phosphorylation of said polypeptide in said sample to a control sample.

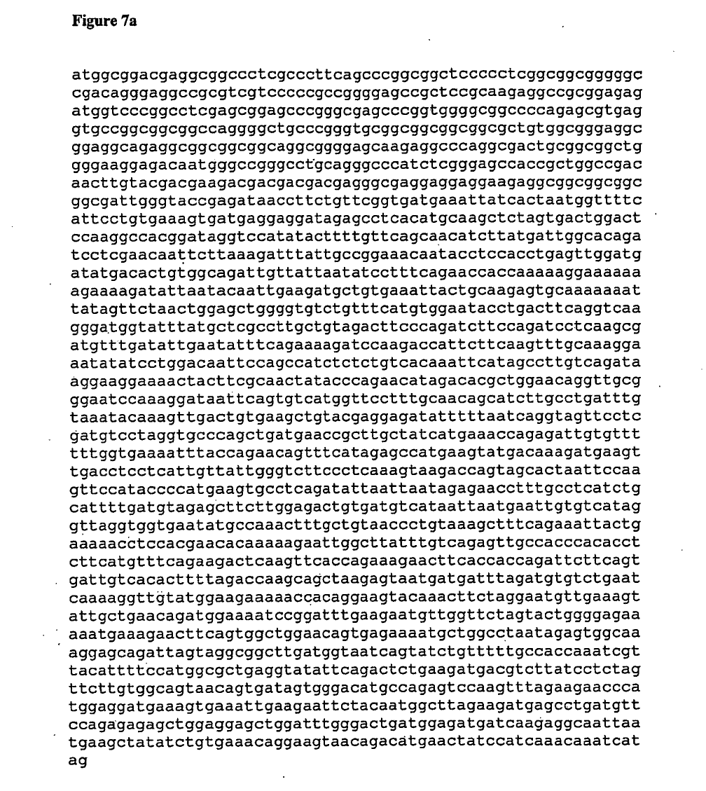

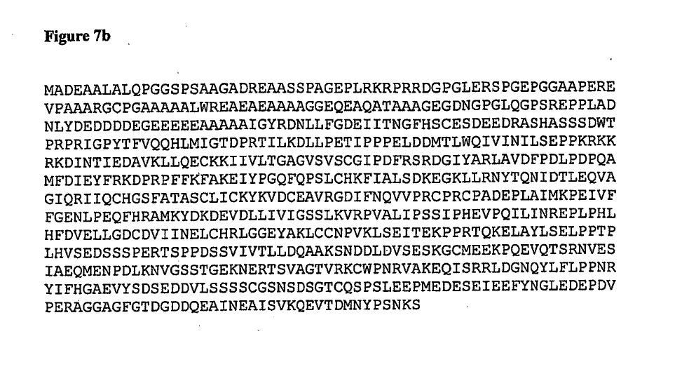

In a preferred method of the invention said sirtuin is SIRT1; preferably said sirtuin is represented by the amino acid sequence in FIG. 7.

In a preferred embodiment of the Invention said phosphorylation is at serine 27 as shown in FIG. 7.

In a further preferred method of the invention said method includes a comparison of the phosphorylation of serine 27 with the phosphorylation of serine 47 in the amino acid sequence represented in FIG. 7.

The detection of phosphorylated amino acids is facilitated by the use of antibodies that specifically recognise phosophorylated serine residues; in particular specific antibodies that bind phosphorylated serine 27 and serine 47 in human SIRT1 are commercially available.

In a preferred method of the invention said disease condition is a hyper-proliferative disease.

In a preferred method of the invention said disease is a viral infection caused by a pathogenic virus.

In a preferred method of the invention said disease is viral induced cancer.

In a preferred method of the invention said disease is a viral induced cancer resulting form a human papilloma virus (HPV).

Human papilloma viruses vary in their pathological effects. For example, in humans so called low risk HPVs such as HPV-6 and HPV-11 cause benign hyperplasia such as genital warts while high risk HPVs, for example HPV-16, HPV-18, HPV-31, HPV-33, HPV-52, HPV-54 and HPV-56 can cause cancers such as cervical and penile carcinoma. HPV-5 and HPV-8 cause malignant squamous cell carcinomas of the skin. HPV-2 is found in malignant and non-malignant lesions in cutaneous and squamous epithelium.

In a preferred method of the invention said human papilloma virus is HPV-16 or HPV-18.

Other viruses that have an association with cancer Include, Polyomavirus which is associated with colorectal cancer; human herpesvirus-8 is associated with Karposi's sarcoma and Epstein Barr Virus associated with Burkitts lymphoma. RNA viruses are also associated with certain cancers, for example Human T cell leukaemia virus-1 and Hepatitis C virus. A yet further example is HIV and Kaposi's sarcoma

In a preferred method of the invention said hyper-proliferative disease is cancer.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers e.g. colorectal cancer, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumour cells form recognizable glandular structures. The term “sarcoma” is art recognized and refers to malignant tumors of mesenchymal derivation.

According to a further aspect of the invention there is provided a method to diagnose and optionally treat a subject suffering from a condition which would benefit from the stimulation of apoptosis with a chemotherapeutic agent comprising the steps of:
i) providing an isolated sample comprising a cell to be tested;
ii) determining the phosphorylation of at least one amino acid residue in a polypeptide comprising an amino acid sequence as represented in FIG. 7;
iii) comparing the phosphorylation of said polypeptide in said sample to a control sample; and
iv) identifying a chemotherapeutic treatment regime that would benefit said subject.

In a preferred method of the invention said sample is additionally tested to determine the p53 genotype of said cell.

In a preferred method of the invention said method includes the administration of at least one chemotherapeutic agent to said subject.

In a preferred method of the invention said chemotherapeutic agent is 5-fluorouracil; preferably 5-fluorouracil is administered with an effective amount of thymidine.

In a preferred method of the invention said phosphorylated amino acid residue is serine 27 in the amino acid sequence represented in FIG. 7.

In a further preferred method of the invention said method includes a comparison of the phosphorylation state of serine 27 with the phosphorylation state of serine 47 in the amino acid sequence represented in FIG. 7.

In a preferred method of the invention said cel/tissue sample comprises pre-cancerous or cancerous cells.

In a preferred method of the invention said pre-cancerous or cancerous tissue comprises transformed epithelial cells.

Preferably said pre-cancerous or cancerous tissue is derived from the colon.

Colorectal cancer is a cancer which occurs in the large intestine and rectum. The colon can be divided into effectively four sections; the ascending colon; the transverse colon; the descending colon; and the sigmoid colon. Most colorectal cancers arise in the sigmoid colon and develop from “polyps” which can grow for several years before becoming cancerous. The early detection of these pre-cancerous growths is obviously desirable since removal of the polyps is a very effective means to stem the progress of disease. There are various types of colorectal cancer. Most cancers of this type are adenocarcinomas which are malignant growths which begin in the epithelial cells which line the colon and rectum. Other cancers of the colon and rectum Include gastrointestinal stromal tumours and lymphomas. In some examples the patient can be asymptomatic and for this reason it Is important that early screening is undertaken to identify those patients in which pre-cancerous polyps are forming. The chemotherapeutic agents typically used to treat colorectal cancer include 5-fluorouracil, leucovorin, irinotecan and capecitabine.

In a preferred method of the invention said method further comprises the administration of a vector that includes a nucleic acid molecule that encodes a p53 polypeptide.

In a preferred method of the invention said nucleic acid molecules are part of an expression vector, preferably an expression vector adapted for eukaryotic gene expression.

The use of viruses or “viral vectors” as therapeutic agents is well known in the art.

Additionally, a number of viruses are commonly used as vectors for the delivery of exogenous genes. Commonly employed vectors include recombinantly modified enveloped or non-enveloped DNA and RNA viruses, preferably selected from baculoviridiae, parvoviridiae, picomoviridiae, herpesveridiae, poxviridae, adenoviridiae, or picornnaviridiae. Chimeric vectors may also be employed which exploit advantageous elements of each of the parent vector properties (See e.g., Feng, et al.(1997) Nature Biotechnology 15:866-870). Such viral vectors may be wild-type or may be modified by recombinant DNA techniques to be replication deficient, conditionally replicating or replication competent.

Preferred vectors are derived from the adenoviral, adeno-associated viral and retroviral genomes. In the most preferred practice of the invention, the vectors are derived from the human adenovirus genome. Particularly preferred vectors are derived from the human adenovirus serotypes 2 or 5. The replicative capacity of such vectors may be attenuated (to the point of being considered “replication deficient”) by modifications or deletions in the El a and/or El b coding regions. Other modifications to the viral genome to achieve particular expression characteristics or permit repeat administration or lower immune response are preferred. In the most preferred practice of the invention as exemplified herein, the vector is replication deficient vector adenoviral vector encoding the p53 tumour suppressor gene A/C/N/53 as described In Gregory, et al, U.S. Pat. No. 5,932,210 issued Aug. 3, 1999 (the entire teaching of which is herein Incorporated by reference).

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein a gene essential for replication of the virus Is under control of a promoter which Is active only in a particular cell type or cell state such that in the absence of expression of such gene, the virus will not replicate. Examples of such vectors are described in Henderson, et al., U.S. Pat. No. 5,698,443; Henderson, et al., U.S. Pat. No. 5,871,726 the entire teachings of which are herein incorporated by reference.

Additionally, the viral genome may be modified to include inducible promoters which achieve replication or expression only under certain conditions. Examples of inducible promoters are known in the scientific literature (see, Yoshida and Hamada (1997) Biochem. Biophys. Res. Comm. 230:426-430; lida, et al. (1996) J. Virol. 70(9):6054-6059; Hwang, et al.(1997) J. Virol 71(9):7128-7131; Lee, et al. (1997) Mol. Cell. Biol. 17(9):5097-5105; and Dreher, et al..(1997) J. Biol. Chem 272(46); 29364-29371.

The viruses may also be designed to be selectively replicating viruses. Particularly preferred selectively replicating viruses are described in Ramachandra, et al. PCT International Publication No. WO00/22137 International Application No. PCT/US99/21452 published Apr. 20, 2000 and Howe, J., PCT International Publication No. WO WO0022136, International Application No. PCT/U.S. 99/21451 published Apr. 20, 2000.

It has been demonstrated that viruses which are attenuated for replication are also useful in gene therapy. For example the adenovirus dl1520 containing a specific deletion in the E1b55K gene (Barker and Berk (1987) Virology 156: 107) has been used with therapeutic effect in human beings. Such vectors are also described in McCormick U.S. Pat. No. 5,677,178 and U.S. Pat. No. 5,846,945.

It may be valuable in some instances to utilize or design vectors to achieve introduction of the exogenous transgene in a particular cell type. Certain vectors exhibit a natural tropism for certain tissue types. For example, vectors derived from the genus herpesviridiae have been shown to have preferential infection of neuronal cells. Examples of recombinantly modified herpesviridiae vectors are disclosed in U.S. Pat. No. 5,328,688. Cell type specificity or cell type targeting may also be achieved in vectors derived from viruses having characteristically broad Infection by the modification of the viral envelope proteins. For example, cell targeting has been achieved with adenovirus vectors by selective modification of the viral genome knob and fibre coding sequences to achieve expression of modified knob and fibre domains having specific interaction with unique cell surface receptors. Examples of such modifications are described in Wickham, et al.(1997) J. Virol 71(11):8221-8229 (incorporation of RGD peptides into adenoviral fibre proteins); Amberg, et al.(1997) Virology 227:239-244 (modification of adenoviral fibre genes to achieve tropism to the eye and genital tract); Harris and Lemoine (1996) TIG 12(10):400-405; Stevenson, et al. (1997) J. Viral. 71(6):4782-4790; Michael, et al (1995) Gene Therapy 2:660-668 (incorporation of gastrin releasing peptide fragment Into adenovirus fibre protein); and Ohno, at al.(1997) Nature Biotechnology 15:763-767 (incorporation of Protein A-IgG binding domain into Sindbis virus).

Other methods of cell specific targeting have been achieved by the conjugation of antibodies or antibody fragments to the envelope proteins (see, e.g. Michael, et al. (1993) J. Biol. Chem 268:6866-6869, Watkins, et al. (1997) Gene Therapy 4:1004-1012; Douglas, et al (1996) Nature Biotechnology 14: 1574-1578. Alternatively, particularly moieties may be conjugated to the viral surface to achieve targeting (see, e.g. Nilson, et al. (1996) Gene Therapy 3:280-286 (conjugation of EGF to retroviral proteins).

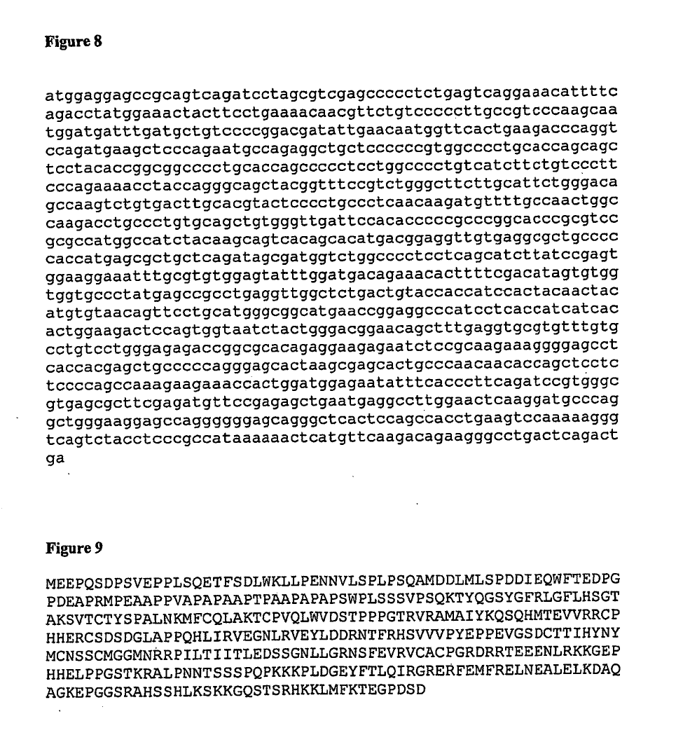

In a preferred method of the invention said nucleic acid molecule encoding p53 is represented by the nucleic acid sequence presented in FIG. 8.

In a preferred method of the invention said nucleic acid molecule encodes a p53 polypeptide as represented by the amino acid sequence in FIG. 9.

In a further preferred method of the invention said subject Is further administered an agent that inhibits the activity of the RNA-binding protein HuR.

In a preferred method of the invention said agent is an antibody or fragment thereof.

In an alternative preferred method of the invention said agent is an siRNA; preferably said siRNA is derived from the nucleic acid sequence in FIG. 10.

In a preferred method of the invention said nucleic acid molecule that encodes p53 is modified wherein said modification prevents or inhibits the binding of the RNA-binding protein HuR to the 5′ and/or 3′ leader sequences of p53 mRNA.

In an alternative preferred method of the invention said subject is administered an agent that inhibits the binding of the RNA-binding protein HuR to mRNA that encodes SIRT1.

According to a yet further aspect of the invention there Is provided a method to screen for agents that modulate the activity of at least one sirtuin associated with the initiation and/or progression of cancer comprising the steps of:
i) forming a preparation comprising at least one sirtuin polypeptide wherein said polypeptide is represented by the amino acid sequence in FIG. 7, or a variant thereof, and at least one candidate agent to be tested; and
ii) determining the activity of said agent with respect to activity of said polypeptide.

In a preferred method of the invention said agent inhibits the phosphorylation of said polypeptide.

In a further preferred method of the invention said polypeptide is represented by the amino acid sequence in FIG. 7.

In a preferred method of the invention said polypeptide is expressed by a cell wherein said cell is transformed or transfected with a nucleic acid molecule that encodes a sirtiun polypeptide. Preferably said nucleic acid molecule is part of a vector adapted for recombinant expression of said nucleic acid molecule. Preferably said vector is provided with a promoter which enables the expression of said nucleic acid molecule to be regulated.

In a preferred method of the invention said cell is derived from the colon, preferably said cell is an epithelial cell which lines the colon.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable, to any other aspect, embodiment or example described herein unless incompatible therewith.

An embodiment of the invention will now be described by example only and with reference to the following figures:

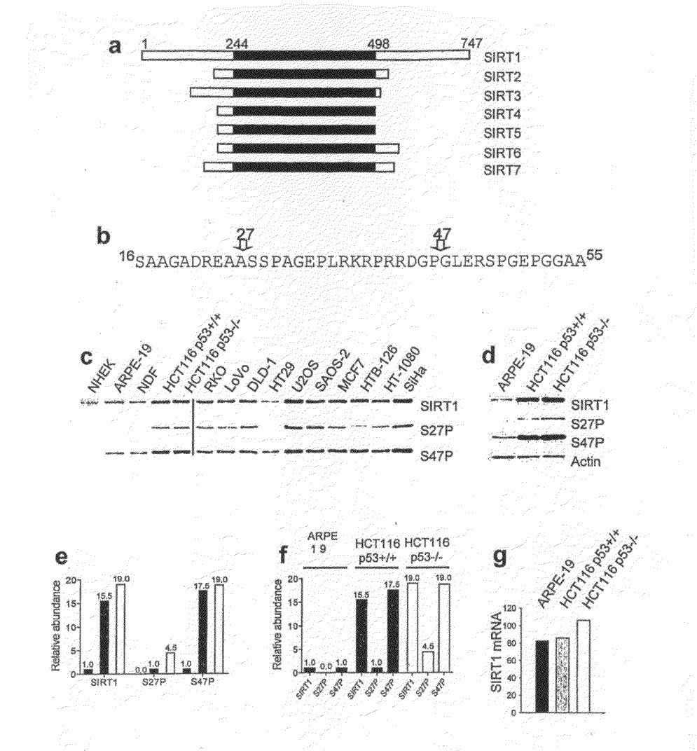

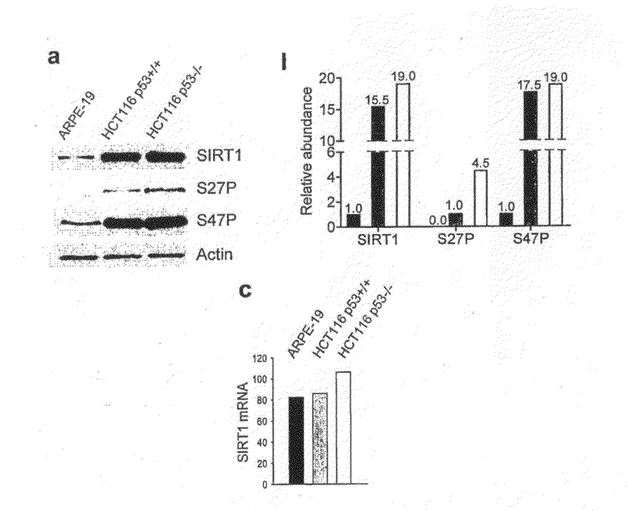

FIG. 1 illustrates cancer cells express elevated levels of SIRT1 protein and this correlates with SIRT1 S27 phosphorylation. a. Schematic of human sirtuins SIRT1-7 showing conserved catalytic domain (black bar) and unique N- and C-terminal domains. b. SIRT1 N-terminal residues 16-55 with sites of phosphorylated serines 27 and 47 indicated. c. Immunoblot showing SIRT1 protein levels, S27P and S47P between 15 human non-cancer (NC) and cancer (C) cell lines. d. Immunoblot showing relative levels of SIRT1, S27P and S47P between non-cancer ARPE-19 cells and isogenic colorectal carcinoma HCT116 p53+/+ and HCT116 p53−/− cells. e, f. Relative levels of total SIRT1, SIRT1 S27P and SIRT1 S47P (Methods), as shown in (d) for ARPE-19 (black bars), HCT116 p53+/+ (grey bars) and HCT116 p53−/− (open bars). g. Relative levels of SIRT1 mRNA between ARPE-19 (black bars), HCT116 p53+/+ (grey bars) and HCT116 p53−/− (open bars);

FIG. 2 illustrates 5-FU induces rapid loss of SIRT1 mRNA associated with p53-dependent decrease in total SIRT1 protein in ARPE-19 and HCT116 cells. a. Levels of SIRT1, laminA/C and GAPDH mRNAs determined by qRT-PCR at 24 h 5-FU exposure. b. SIRT1 mRNA levels at Oh, 2 h, 6 h, 12 h and 24 h after treatment with 5-FU. c. Protein levels of total SIRT1, SIRT1 S27P, SIRT1 S47P, p53 and p53 K382Ac at Oh, 2 h, 6 h, 12 h and 24 h after treatment with 5-FU. d. Phase contrast images of ARPE-19, HCT116 p53+/+ and HCT116 p53−/− cells after 24 h 5-FU. e. Relative numbers of apoptotic cells Identified by annexin-V staining after 24 h 5-FU;

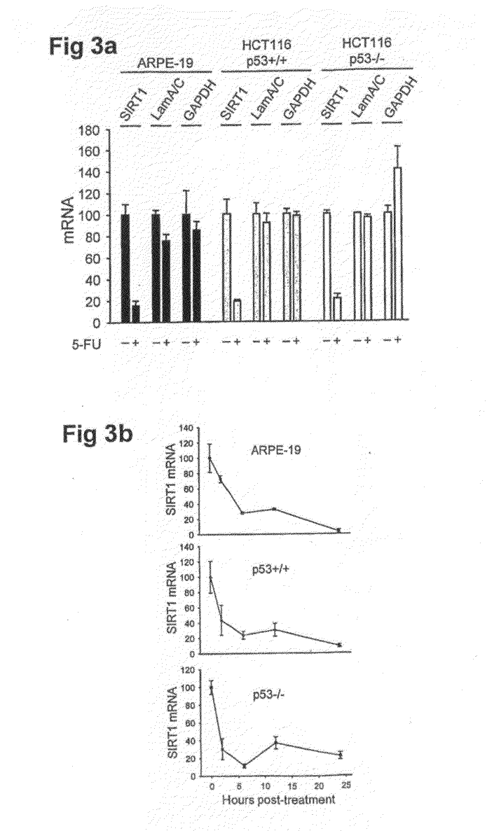

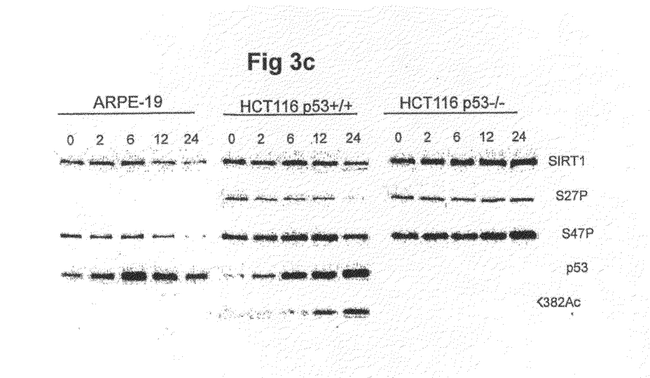

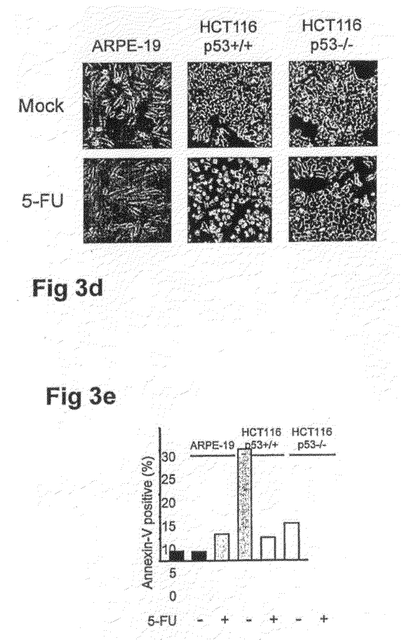

FIG. 3 illustrates that in cancer cells the maintenance of SIRT1 protein and phosphorylation levels is dependent upon JNK2 and p53. a. SIRT1, JNK1 and JNK2 protein levels and b. mRNA levels in ARPE-19 cells at 24 h, 48 h, 72 h and 96 h post-transfection with SIRT1 siRNA, JNK2 siRNA or JNK1 siRNA as indicated. c-e. Immunoblots showing SIRT1, p53, JNK1 and JNK2 proteins in (c) non-cancer ARPE-19 cells, (d) HCT116 p53+/+ cells and (e) HCT116 p53−/− cells after JNK1 and JNK2 silencing and treatment with 5-FU+U (see text). f. SIRT1 mRNA levels in HCT116 p53+/+ cells after 24 h treatment with 5-FU or 5-FU+U;

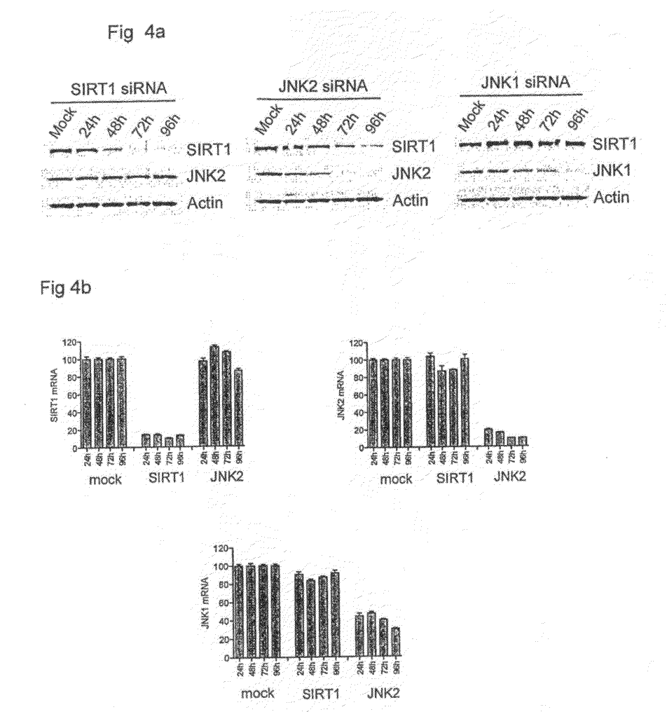

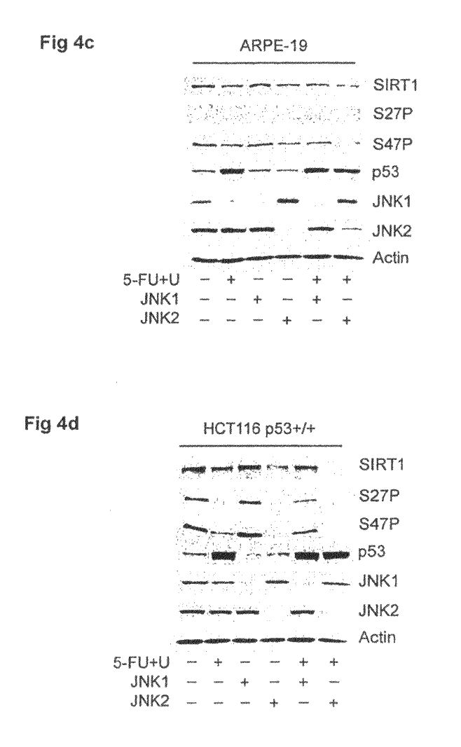

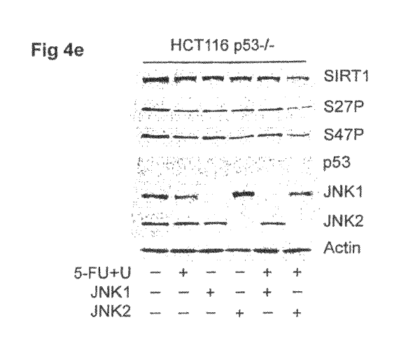

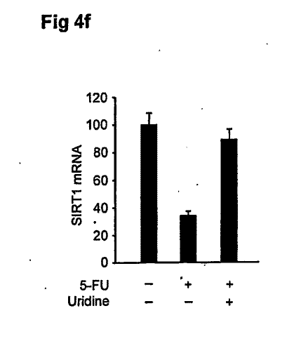

FIG. 4 illustrates a schematic summarising SIRT1 phosphorylation in non-cancer and cancer cells, and the effects of 5FU and/or JNK2 silencing;

FIG. 5a is the nucleic acid sequence of human JNK2 variant 1; FIG. 5b is the nucleic acid sequence of human JNK2 variant 2; FIG. 5c is the nucleic acid sequence of human JNK2 variant 4;

FIG. 6a is the amino acid sequence of human JNK2 variant 1; FIG. 6b is the amino acid sequence of human JNK2 variant 2; FIG. 6c is the amino acid sequence of human JNK2 variant 4;

FIG. 7a is the nucleic acid sequence of human SIRT1; FIG. 7b is the amino acid sequence of human SIRT1;

FIG. 8 is the nucleic acid sequence of human p53;

FIG. 9 is the amino acid sequence of human p53;

FIG. 10a is the nucleic acid sequence of the RNA-binding protein HuR; FIG. 10b is the amino acid sequence of RNA-binding protein HuR;

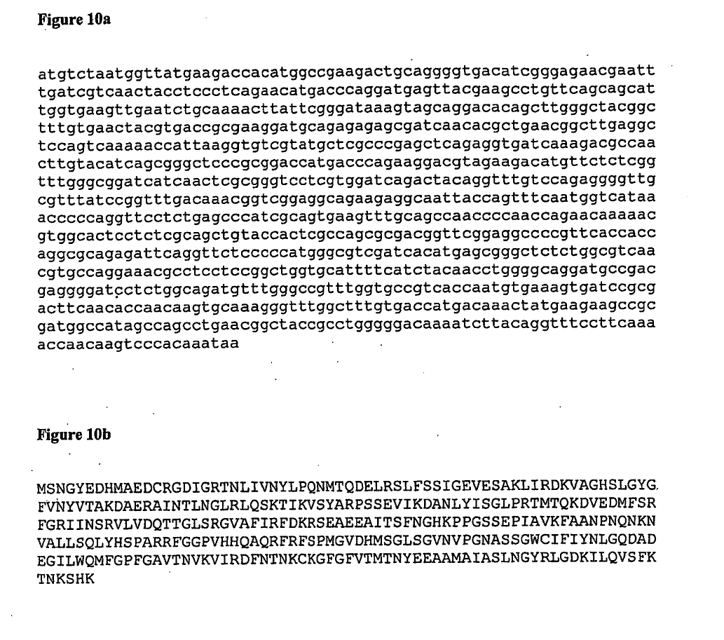

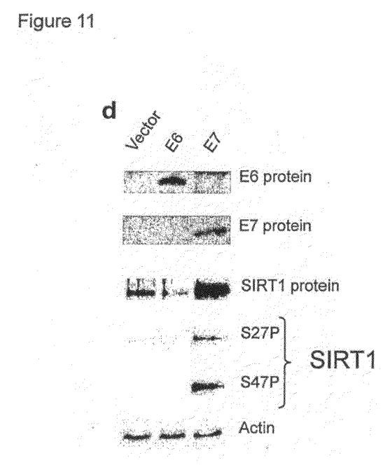

FIG. 11 is a western blot showing expression in human keratinocytes tranfected with exp ression vectors for HPV16 E6 and HPV16 E7 oncogenes and that E7 Induces differential phosphorylation of SIRT1;

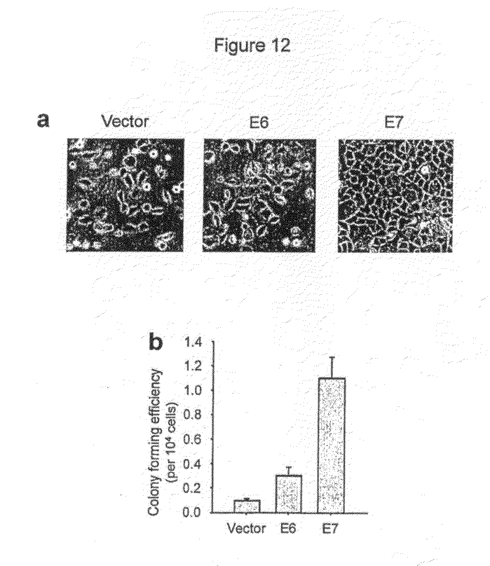

FIG. 12 Phenotype of enforced expression of HPV-16 viral transcripts in NHEK cells. a. Digital phase contrast images of NHEK cells at 48 hours post-transfection with E6 or E7 viral transcript expression vectors. b. Colony forming assay of NHEK transfected with E6 or E7 expression vectors; and

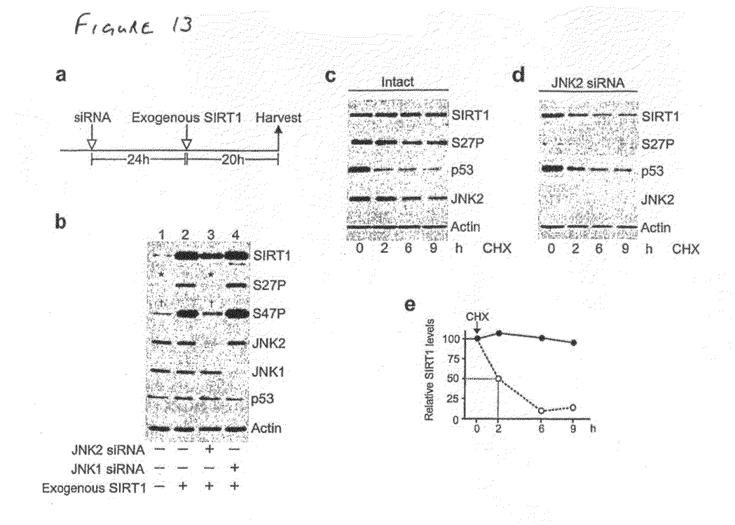

FIG. 13 JNK-dependent phosphorylation at S27 stabilises exogenous SIRT1 protein. a. Schematic of experimental protocol for RNA′ followed by transfection with exogenous SIRT1 expression vector (see text). b. Exogenous expression of SIRT1 in intact HCT116 cells (lane 2), JNK2-depleted cells (lane 3) and JNK1-depleted cells (lane 4) detected by immunoblotting. Lane 1 =intact, non-transfected cells. Note that in both intact cells and JNK1-depleted cells the exogenous SIRT1 is phosphorylated on S27 and S47, whereas in JNK2-depleted cells S27P levels reflect endogenous SIRT1 (lanes 1 and 3, asterix), c, d. Time course analyses of SIRT1 protein levels and SIRT1 S27P following addition of cycloheximide in intact (panel c) and JNK2-depleted (panel d) HCT116 cells as indicated. e. SIRT1 protein levels at indicated times post treatment with cycloheximide (CHX) in intact cells (solid circles, solid line) and in JNK-depleted cells (open circles, dashed line).

### Materials and Methods

### Cell Lines, Transfection and Drug Treatments

Epithelial cancer cell lines RKO, LoVo, DLD-1, HT29, U2OS, SAOS-2, MCF7, HTB-126, HT-1080 and SiHa were obtained from ATCC. Isogenic colorectal carcinoma cell lines HCT116 p53+/+ and HCT116 p53−/− were a kind gift of Bert Vogelstein. Non-cancer cells were normal diploid fibroblasts (NDF) and ARPE-19 immortalised retinal epithelial cells (ATCC), and normal human embryonic keratinocytes (NHEK, Invitrogen). Cells were cultured according to the supplier's protocols. siRNA sequences, transfection protocols and target validations were as described (Ref. 27 and Methods). For drug treatments, cells were treated 48 hours post-plating with final concentration of 5-FU (375 OM) and uridine (425 OM) as indicated and harvested 24 h later. For combined transfection/drug treatment, drugs were applied 24 h post-transfection and cells harvested 24 h later. For time course analyses cells were harvested at indicated time points post-treatment.

Quantification of mRNA

Cellular mRNAs were extracted for quantitative RT-PCR as previously described²⁷. Primers and thermal cycling conditions were as described²⁷ (Methods); quantifications were performed in triplicate.

### Immunoblotting

Cells were prepared as described (Methods). For immunoblotting equivalent numbers of cells, or equivalent amounts of protein, were loaded in each lane. Antibodies were: Anti-SIRT1 (H-300, Santa Cruz), anti-phosphoserine27-SIRT1 (#2327, Cell Signaling), anti-phosphoserine47-SIRT1 (#2314, Cell Signaling), anti-actin (MAB1501, Chemicon), anti-p53 (00-1, Santa Cruz), anti-acetylated-p53-lysine382 (#2525, Cell Signaling), anti-JNK1 (F-3, Santa Cruz), anti-JNK2 (#4672, Cell Signaling). Visualisation of bound antibodies was by ECL, with quantitation by densitometry of signals within the linear range.

### Microscopy and Determination of Apoptosis

Phase contrast digital images were captured using an Axiovert 200M microscope (Zeiss). Apoptotic cells were identified by flow cytometry using annexin-V-Fluos (Roche) following the manufacturer's protocol.

### Cell Culture

Cells were cultured at 37° C. in 5% CO₂. Colorectal carcinoma cell lines HCT116 (p53+/30 and isogenic p53−/−), LoVo (p53 wild-type) and HT29 (p53 mutant) were cultured in DMEM (Gibco); colorectal carcinoma cell line DLD-1 (p53 mutant) was cultured in RPMI 1640 (Gibco) supplemented with 1mM sodium pyruvate; colorectal carcinoma cell line RKO (p53 wild-type) was cultured in MEM (Gibco); osteogenic sarcoma cell lines U2OS (p53 wild-type) and SAOS-2 (p53 null) were cultured in McCoy's 5A modified (ATCC); fibrosarcoma cell line HT-1080 (p53 wild-type) was cultured in EMEM (ATCC); breast carcinoma cell line MCF7 (p53 wild-type) was cultured in EMEM (ATCC) supplemented with 10 μg insulin; breast carcinoma cell line HTB-126 (p53 mutant) was cultured in DMEM (ATCC) supplemented with 10 μg insulin; human papilloma virus type 16-transformed cervical carcinoma cell line SiHa (p53 wild-type) was cultured in EMEM (ATCC) supplemented with 1 mM sodium pyruvate and 0.1 mM nonessential amino acids; spontaneously immortalised non-cancer retinal epithelial cell line ARPE-19 was cultured in DMEM:F12 (Gibco); normal diploid lung fibroblast (NDF) WI-38 was cultured in MEM (Gibco) supplemented with 1 mM sodium pyruvate and 0.1 mM nonessential amino acids. All media were supplemented with 10% foetal bovine serum, and either 2 mM L-glutamine (LoVo, HT29, DLD-1, RKO, HT-1080, MCF7, SiHa, WI-38) or 4 mM L-glutamine (HCT116, U2OS, SAOS-2, HTB-126, ARPE-19). Normal human epidermal keratinocytes (NHEK, Invitrogen) were cultured in defined serum-free media (KSF) in collagen-IV (Sigma) coated flasks. For experiments, cell lines were used between passage 2-4 following removal from liquid nitrogen.

### Transfection and Drug Treatments

Cells for transfection and/or drug treatment were plated at a density of 6×10⁴ (ARPE-19) or 1.4×10⁵ (HCT116) per well in 6-well plates. For the experiment in FIG. 3a, ARPE-19 cells were plated at a density of 4×10⁴ per well. Transfections were performed 24 hours post-plating in Opti-MEM (Gibco) using Oligofectamine (Invitrogen) according to the manufacturer's instructions. Chemically synthesized, HPLC-purified siRNAs were obtained from Qiagen or Dharmacon. SIRT1 siRNA sequence has been described²⁷. JNK1 siRNA sequences were: Sense 5′-CUCCACCACCAAAGAUCCC(dTdT)-3′, antisense 5′-GGGAUCUUUGGUGGUGGAG(dTdT)-3′. JNK2 siRNA sequences were: Sense 5′-GAGCUGGUGAAAGGUUGUG(dTdT)-3′, antisense 5′-CACAACCUUU CACCAGCUC(dTdT)-3′. Specificities of SIRT1 siRNA, JNK1 sIRNA and JNK2 siRNA have been validated previously¢ (Ahmed and Milner, in submission). Cells were harvested for analysis 48 h post-transfection, except where indicated otherwise. For drug treatment, drugs were applied by complete media replacement 48 h post-plating at final concentration 375 μM (5-FU) or 425 μM (uridine). Cells were harvested for analysis 24h following application of drugs, or at the indicated timepoints for time-course analysis. For combined transfection/drug experiments, drugs were applied approximately 20 hours post-transfection. Cells from combined transfection/drug experiments were harvested for analysis after 24 hours of drug exposure (˜48 hours post-transfection).

### Protein Preparation and Immunoblotting

For preparation of total protein lysates, cell counts or protein assay was performed, with the method chosen consistent within each experiment. Cells were lysed in lysis buffer IPAMN (10 mM TRIS base pH 8.0, 140 mM NaCl, 2 mM CaCl₂, 0.5% NP-40, 5 U ml⁻¹ micrococcal nuclease) for 3 minutes at room temperature followed by addition of 0.25× volumes of 6×SDS-PAGE sample buffer and brief heating to 90° C. Samples for protein assay were withdrawn before addition of 6×SDS-PAGE sample buffer, and assayed using BCA Protein Assay Kit (Pierce) according to the manufacturers instructions. Equivalent numbers of cells, or equivalent amounts of protein, were electrophoresed on 10% or 15% SDS-PAGE, electrobiotted to Protran membrane (Schleicher&Schuell) using a wet-blot transfer system, blocked for 1 hour at room temperature and probed with primary antibodies overnight at 4° C. Antibodies were: Anti-SIRT1 (H-300, Santa Cruz), anti-phosphserine27-SIRT1 (#2327, Cell Signaling), anti-phosphoserine47-SIRT1 (#2314, Cell Signaling), anti-actin (MAB1501, Chemicon), anti-p53 (DO-1, Santa Cruz), anti-acetylated-p53-lysine382 (#2525, Cell Signaling), anti-JNK1 (F-3, Santa Cruz), anti-JNK2 (#4672, Cell Signaling). Bound antibodies were visualised by ECL (Roche). Densitometry was performed from under-exposed images within the linear range, using Quantity One analysis software (BioRad).

### Quantitative RT-PCR

Relative mRNA levels were quantitated by realtime RT-PCR. Primer sequences and thermal cycles for GAPDH and LaminA/C have been described previously²⁷. SIRT1 primers 5′-CTAATTCCAAGTTCCATACCC-3′ and 5′-CTGAAGAATCTGGTGGTGAAG-3′ were used in the thermal cycle: 50° C. for 30 minutes, 94° C. for 15 minutes, followed by 35 cycles of 94° C. for 30 seconds, 55° C. for 30 seconds, 72° C. for 30 seconds, then 75° C. for 15 seconds before the plate was read. JNK1 primers 5′-CCAGGAAGGGACTATATTGATC-3′ and 5′-TCTCTCCTCCAAGTCCATAACT-3′ were used in the thermal cycle: 50° C. for 30 minutes, 94° C. for 15 minutes, followed by 35 cycles of 94° C. for 30 seconds, 57° C. for 30 seconds, 72° C. for 30 seconds, then 75° C. for 15 seconds before the plate was read. JNK2 primers 5′-GAAGCCTAGCAACATTGTTG-3′ and 5′-GATCAATATGGTCAGTGCCT-3′ were used in the thermal cycle: 50° C. for 30 minutes, 94° C. for 15 minutes, followed by 35 cycles of 94° C. for 30 seconds, 55° C. for 30 seconds, 72° C. for 30 seconds, then 75° C. for 15 seconds before the plate was read. All RT-PCR quantifications were performed in triplicate.

### Microscopy and Determination of Apoptosis

Digital phase contrast images of cells were taken on an Axiovert 200M microscope. Apoptotic cells were identified using Annexin-V-Fluos kit (Roche) with analysis by CeliQuest software on FACsCalibur (BD).

### EXAMPLE 1

We have asked if SIRT1 is subject to post-translational regulation. Putative phosphorylation sites at S27 and S47 within the unique SIRT1 N-terminal domain (FIGS. 1a and b) have previously been revealed by mass spectrometry. To investigate the significance of these sites we screened a series of human cell lines using commercially available antisera raised against synthetic SIRT1 S27P and S47P phosphopeptides (Methods). SIRT1 knock-down by RNA Interference (RNAi) showed equivalent knock-down of S27P and S47P in HCT116 cells, thus validating anti-SIRT1 activity of the anti-phosphopeptide antisera (FIG. 1c). This result also demonstrates that both SIRT1 S27P and S47P remain detectable even when cellular levels of total SIRT1 are reduced>30-fold (as determined by gel scanning, Methods).

Under basal conditions of culture SIRT1 S27P appeared restricted to cancer cells, being undetectable in non-cancer ARPE-19 epithelial cells, primary human keratinocytes (NHEK) and normal diploid fibroblasts (NDFs) (FIG. 1c). In contrast SIRT1 S47P was evident in both non-cancer and cancer cell lines (with the exception of primary human epidermal keratinocytes in which S47P was undetectable; FIG. 1c). S47P levels tended to parallel total SIRT1 protein (SIRT1:S47P ratio range 0.8 to 1.5, as determined by gel scanning, see Methods) suggesting constitutive phosphorylation at this site. In contrast S27P detection was variable relative to total SIRT1 in the different cell lines (FIG. 1c; SIRT1:S27P ratio range 3.4 to 34). The observed variability in S27 phosphorylation relative to total SIRT1 protein indicates that S27 phosphorylation is subject to cellular regulation.

Total SIRT1 protein levels were generally high in cancer relative to non-cancer cells (FIG. 1c; see also below). The one exception was HT29 colorectal cancer cells which expressed low levels of total SIRT1. The HT29 cells also differed from the remaining cancer cell lines in that SIRT1 was un-phosphorylated on S27 (FIG. 1c). Thus S27P is variable relative to total SIRT1 and correlates with the elevated SIRT1 protein levels observed in cancer cells under basal conditions of growth.

More detailed comparison of SIRT1 protein levels In non-cancer versus cancer cell lines employed ARPE-19 cells (spontaneously immortalised human retinal epithelial cells of non-cancerous origin) and HCT116 p53+/+ and p53−/− isogenic human colorectal cancer epithelial cells²⁴. Comparison of SIRT1 protein levels on a per cell basis revealed ˜15 to 20-fold higher SIRT1 levels in HCT116 cells compared with ARPE-19 cells (FIG. 1d, 1e & 1f). S47P paralleled SIRT1 levels, whilst the S27P:SIRT1 ratio was approximately four-fold higher in HCT116 p53−/− cells compared with HCT116 p53+/+ cells (FIG. 1e & 1f). This suggests that the presence of p53 may influence phosphorylation of SIRT1 at S27 (see also below). Importantly SIRT1 mRNA levels were similar in both ARPE-19, HCT116 p53+/+ and HCT116 p53−/− cells (FIG. 1g). Thus the observed differences in SIRT1 protein levels between ARPE-19 and HCT116 cells were not attributable to differences in gene expression levels or mRNA stability. They may therefore reflect differences in the efficiency of mRNA translation and/or SIRT1 protein stability. However, since SIRT1 is not thought to be regulated via variable translation efficiency, our results indicate that high SIRT1 protein levels in human cancer cells reflect abnormally increased protein stability. Since SIRT1 protein stability shows a positive correlation with phosphorylation at S27 it is possible that S27P prolongs the half-life of cellular SIRT1 protein.

### EXAMPLE 2

We next asked if 5-FU induces any changes in SIRT1 levels and/or phosphorylation status. 5-FU is a clinically important anti-cancer drug and is the treatment of choice for colorectal cancer. The mechanism of action of 5-FU Is complex and includes perturbation of DNA synthesis culminating in DNA damage, and also incorporation into newly synthesised RNAs with consequential Inhibition of RNA processing^{25, 26}. In the present study treatment of cells with 5-FU for 24 h resulted in 80% depletion of SIRT1 mRNA, whilst lamin NC and GAPDH mRNA levels were relatively unaffected (FIG. 2a). More detailed time-course analysis revealed a rapid loss of SIRT1 mRNA following administration of 5-FU, with estimated t_{1/2}<2 h in HCT116 cells and t_{1/2}˜4 h in ARPE-19 cells (FIG. 2b). The normal half-life of SIRT1 mRNA (˜8 h) is known to be dependent upon HuR-SIRT1 mRNA complexing^{14,15}. In the absence of HuR the half life of SIRT1 mRNA falls to around 1.5 h. Moreover, oxidative stress triggers dissociation of [HuR-SIRT1 mRNA] complexes and thus promotes SIRT1 mRNA decay. Our own independent results now indicate that the anti-cancer drug 5-FU also promotes SIRT1 mRNA decay (see above and FIG. 2b). Future studies will explore the potential involvement of HuR in this effect. For the purposes of the present study the loss of SIRT1 mRNA is an important backdrop for interpreting the effects of 5-FU upon SIRT1 phosphorylation and SIRT1 protein stability.

In ARPE-19 cells the levels of SIRT1 protein increased 6 h after 5-FU treatment and subsequently declined by 24 h (FIG. 2c, left hand panel). Cellular SIRT1 protein levels in 5-FU-treated HCT116 p53+/+ cells were stable over 12 h, but declined by 24 h post-treatment (FIG. 2c, centre panel). Reduced SIRT1 protein levels correlated with loss of S27 phosphorylation (FIG. 2c, centre panel). Importantly, comparison with isogenic HCT116 p53−/− cells revealed that, in the absence of p53, the level of SIRT1 protein remains constant up to at least 48 h post treatment with 5-FU (FIG. 2c, right hand panel, 48 hr data not shown). In addition, phosphorylation at S27 (lost in HCT116 p53+/+cells) was retained In HCT116 p53−/− cells exposed to 5-FU (FIG. 2c, right hand panel). These observations Identify SIRT1 as a regulable target of the anti-cancer drug 5-FU and also implicate p53 as an essential player linking 5-FU-induced effects with SIRT1 S27 phophorylation/protein stability. Thus p53 is involved in the regulation of SIRT1 protein turnover in the presence of 5FU.

In both ARPE-19 and HCT116 cells the drug 5-FU induces a p53 response with p53 protein stabilisation and up-regulation of p53 target genes p21 and HDM2 (FIG. 2c and data not shown). SIRT1 Is known to deacetylate activated p53 in mouse embryonic fibroblasts and thus down-regulate the p53 response¹¹. This is entirely consistent with our present results using human epithelial cells (ARPE-19) in which the p53 response was attenuated and acetylation of p53 was undetectable (FIG. 2c, left hand panel). The cells remained viable (FIGS. 2d, 2e). In contrast HCT116 p53+/+ colorectal cancer cells displayed a sustained p53 response to 5-FU and this correlated with induction of p53 K382 acetylation and apoptosis (FIGS. 2c centre panel and 2d, 2e). Similar differences between normal and cancerous cells may contribute to the preferential anti-cancer effects of 5-FU in the clinic.

### EXAMPLE 3

Previously we have observed that selective silencing of JNK2 by RNA interference (RNAi) results in reduced levels of SIRT1 protein (Ahmed and Milner, unpublished observations and FIG. 3a centre panel). This unexpected effect was attributed to decreased SIRT1 protein stability since SIRT1 mRNA levels remained constant (FIG. 3a and b, centre panels). The effect was specific to JNK2 since JNK1 silencing had no effect upon SIRT1 protein levels (FIG. 3a and b, right hand panels). Also there was no reciprocal effect of SIRT1 silencing on JNK2 protein levels (FIG. 3a, left hand panel). In the light of our present observations we considered that the JNK2 kinase may, directly or indirectly, influence SIRT1 protein stability and that such an effect may operate via SIRT1 phosphorylation.

Depletion of JNK1 by RNAi had no effect upon SIRT1 protein levels nor upon S27 or S47 phosphorylation in ARPE-19, HCT116 p53 +/+ or HCT116 p53−/− cells (FIGS. 3c-e). In contrast depletion of JNK2 in HCT116 p53 +/+ cells resulted in loss of SIRT1 S27P, reduced SIRT1 protein levels and barely detectable S47P (FIG. 3d). These effects on SIRT1 protein were evident in the absence of any reduction in SIRT1 mRNA levels (not shown, results similar to those observed for ARPE19 cells FIG. 3b, middle panel). In parallel cultures cells were treated with 5-FU in the presence of excess uridine (5-FU+U) which rescues SIRT1 mRNA levels (FIG. 3f). Thus we were able to compare the effects of 5-FU and of JNK2 silencing whilst maintaining SIRT1 mRNA levels. In HCT116 p53+/+ cells 5-FU+U alone caused loss of S27P accompanied by partial depletion of total SIRT1 protein paralleled by S47P decrease (FIG. 3d). Strikingly, 5-FU+U treatment of JNK2-depleted cells resulted in total loss of SIRT1 protein (FIG. 3d). The cells died by apoptosis.

In HCT116 p53−/− cells JNK2 silencing or treatment with 5-FU+U had little effect on SIRT1 protein levels or phosphorylation status (FIG. 3e). This indicates that the above observations with HCT1116 p53+/+ cells (FIG. 3d) are p53-dependent. Combined 5-FU plus JNK2 silencing induced only partial reduction of SIRT1 and total protein, S27P and S47P remained detectable (FIG. 3e). Similarly in ARPE-19 cells total SIRT1 levels and S47P were also only partially responsive to JNK2 depletion and 5-FU+U (FIG. 3c). The cells also remained viable. Thus although ARPE-19 cells are positive for p53 their SIRT1 status proved relatively resistant to JNK2 depletion and 5-FU+U treatment.

Given their diverse roles in health and disease the sirtuins are recognised as important targets for therapeutic manipulation. Our discovery of post-translational regulation via the variable N-terminal sirtuin domain Identifies a route for selective therapeutic targeting of SIRT1 without compromising normal functioning of other sirtuins. Moreover the observed link between S27 phosphorylation and abnormal SIRT1 protein accumulation in cancer cells identifies SIRT1 phosphorylation as a promising target for the development of novel anti-cancer therapies.

### EXAMPLE 4

FIG. 11 illustrates expression levels and phosphorylation status of SIRT1 protein in primary human keratinocytes following expression of vectors encoding HPV16 E6 and HPV16 E7 oncoproteins. Cells were transfected with expression vectors and analysed 48 h post-transfectlon. Immunoblots showing detection of HPV E6, HPV E7, total SIRT1, SIRT1 S27P and SIRT1 S47P as indicated down right hand side of panels. Actin loading control is also shown. Lanes: vector =vector only control transfection; E6=HPV16 E6 expression vector; E7=HPV16 E7 transfection vector.

### EXAMPLE 5

To further explore the relationship between SIRT1 and JNK2 we expressed exogenous . SIRT1 in HCT116 p53+/+ cells in which JNK2 had been depleted by RNAI (Methods and FIG. 13a). When expressed in intact cells exogenous SIRT1 protein was phosphorylated at both S27 and S47 (FIG. 13b, lane 2). However, when expressed in JNK2-depleted cells the exogenous SIRT1 protein appeared non-phosphorylated at S27: only endogenous SIRT1 S27P was detectable (FIG. 13b, lanes 1 and 3, asterix). In addition total exogenous SIRT1 protein levels were reduced in JNK2-depleted cells (FIG. 13b, lane 3 cp. lane 2, SIRT1), consistent with reduced SIRT1 protein stability in the absence of S27 phosphorylation. In JNK2-depleted cells exogenous SIRT1 S47P was clearly detectable (FIG. 13b lanes 1 and 3, dagger), albeit at reduced levels relative to those in intact cells (FIG. 13b, lanes 2 and 3) indicating that S27P can influence SIRT1. S47 kinase/phosphatase activity. When JNK1 was silenced by RNAi instead of JNK2 the exogenous expression of SIRT1 and its phosphorylation were indistinguishable from the intact controls (FIG. 13b, cp. lanes 2 and 4). Thus JNK2, but not JNK1, is required for S27 phosphorylation and associated accumulation of SIRT1 protein.

The above results indicate that depletion of JNK2 results in the inability of HCT116 cells to phosphorylate newly expressed SIRT1 protein at S27. Reduced accumulation of SIRT1 protein under these conditions is consistent with the premise that JNK2-dependent phosphorylation of SIRT1 at S27P stabilises the SIRT1 protein. This was confirmed by time course studies in which exogenous SIRT1 protein levels were monitored following inhibition of protein synthesis by cycloheximide. As expected in both intact (FIG. 13c) and in JNK2-depleted cells (FIG. 13d) the p53 protein exhibited a short half-life (<2 h) following addition of cycloheximide, whilst actin protein levels were stable over 9 h under both conditions (FIG. 13c, d). In intact cells exogenous SIRT1 protein was phosphorylated at S27 (FIG. 13c) and was also stable over 9 hr (FIGS. 13c and 13e). However, in JNK2-depleted cells exogenous SIRT1 protein lacked S27P (FIG. 13d) and protein levels declined with a half life of approximately 2 hrs (FIGS. 13d and 13e). We conclude that JNK2 is required, directly or indirectly, for phosphorylation of SIRT1 at S27 and that this is linked with stabilisation of SIRT1 protein in HCT116 cancer cells.

### REFERENCES

1. McBurney, M. W. et al. The mammalian SIR2alpha protein has a role in embryogenesis and gametogenesis. Mol. Cell Biol. 23, 38-54 (2003).
2. Cheng, H. L. et al. Developmental defects and p53 hyperacetylation in Sir2 homolog (SIRT1)-deficient mice. PNAS USA 100, 10794-10799 (2003).
3. Rodgers, J. T. et al. Nutrient control of glucose homecistasis through a complex of

PGC-1 alpha and SIRT1. Nature 434, 113-118 (2005).
4. Hallows, W. C., Lee, S. & Denn, J. M. Sirtuins deacetylate and activate mammalian acetyl-CoA synthetases. PNAS USA 103, 10230-10235 (2006).
5. Brunet, A. et al. Stress-dependent regulation of FOXO transcription factors by the SIRT1 deacetylase. Science 303, 2011-2015 (2004).
6. Chua, K. F. et al. Mammalian SIRT1 limits replicative lifespan in response to chronic genotoxic stress. Cell Metab. 2, 67-76 (2005).
7. Qiu, W. et al. Neuronal SIRT1 activation as a novel mechanism underlying the prevention of Alzheimer disease amyloid neuropathology by calorie restriction. J. Biol. Chem. 281, 21745-21754 (2006).
8. Yuan, Z., Zhang, X., Sengupta, N., Lane, W. S. & Seto, E. SIRT1 regulates the function of the Nijmegen breakage syndrome protein. Mol. Cell 27, 149-162 (2007).
9. Imai, S., Armstrong, C. M., Kaeberlein, M. & Guarente, L. Transcriptional silencing and longevity protein Sir2 is an NAD-dependent histone deacetylase. Nature 403, 793-800 (2000).
10. Blander, G. & Guarente, L. The SIR2 family of protein deacetylases. Annu. Rev. Biochem. 73, 417-435 (2004).
11. Langley, E. et al. Human SIR2 deacetylates p53 and antagonizes PML/p53induced cellular senescence. EMBO J. 21, 2382-96 (2002).
12. Chen, W. Y. et al. Tumor suppressor HIC1 directly regulates SIRT1 to modulate p53-dependent DNA-damage responses. Cell 123, 437-448 (2005).
13. Zhang, Q. et al. Metabolic regulation of SIRT1 transcription via a HIC1:CtBP corepressor complex. PNAS USA 104, 829-833 (2007).
14. Abdelmohsen, K et al. Phosphorylation of HuR by Chk2 regulates SIRT1 expression. Mol. Cell 25, 543-557 (2007).
15. Wilusz, C. J. & Wilusz, J. HuR-SIRT1: The hairy world of posttranscriptional control. Mol. Cell 25, 485-487 (2007).
16. Vaquero, A. et al. Human SirT1 interacts with histone H1 and promotes formation of facultative heterochromatin. Mol. Cell 16, 93-105 (2004).
17. Senawong, T. et al. Involvement of the histone deacetylase SIRT1 in chicken ovalbumin upstream promoter transcription factor (COUP-TF)-interacting protein 2-mediated transcriptional repression. J. Biol. Chem. 278, 43041-43050 (2003).
18. Senawong, T., Peterson, V. J. & Leid, M. BCL11A-dependent recruitment of SIRT1 to a promoter template in mammalian cells results in histone deacetylation and transcriptional repression. Arch. Biochem. Biophys. 434, 316-325 (2005).
19. Luo, J. et al. Negative control of p53 by Sir2a promotes cell survival under stress. Cell 107, 137-148 (2001).
20. Vaziri, H. et al. hSIR2(SIRT1) functions as an NAD-dependent p53 deacetylase. Cell 107, 149-159 (2001).
21. Barak, Y., Juven, T., Haffner, R. & Oren, M. Mdm-2 expression is induced by wild-type p53 activity. EMBO J 12, 461-468 (1993).
22. Moll, U. M. & Petrenko, 0. The MDM2-p53 interaction. Mol. Cancer Res. 1, 1001-1008 (2003).
23. Beausoleil, S. A. et al. Large-scale characterisation of HeLa cell nuclear phosphoproteins. PNAS USA 101, 12130-12135 (2004).
24. Bunz, et al., Disruption of p53 in human cancer cells alters the responses to chemotherapeutic agents. J. Clin. Invest. 104, 263-269 (1999).
25. Noordhuis, P. et al. 5-Flouorouracil Incorporation into RNA and DNA in relation to thymidylate synthase inhibition of human colorectal cancers. Annals Oncol. 15, 1025-1032 (2004).
26. Longley, D. B., Harkin, D. P. & Johnston, P. G. 5-Fluorouracil: mechanisms of action and clinical strategies. Nat. Rev. Cancer 3, 330-38 (2003).
27. Ford, J., Jiang, M. & Milner, J. Cancer-specific functions of SIRT1 enables human epithelial cancer cell growth and survival. Cancer Res. 65, 10457-10463 (2005).

1. A combined therapeutic composition comprising: an inhibitor of JNK2 and at least one chemotherapeutic agent.

2. A composition according to claim 1 wherein JNK2 is encoded by a nucleic acid molecule comprising a nucleic acid sequence as shown in SEQ ID NO: 1-3 or a nucleic acid molecule that hybridizes under stringent hybridization conditions to the sequence in SEQ ID NO: 1-3 and that encodes a polypeptide which has the activity of JNK2.

3. A composition according to claim 2 wherein JNK2 is encoded by a nucleic acid molecule as represented in SEQ ID NO: 1-3.

4. A composition according to claim 2 wherein said nucleic acid molecule encodes a polypeptide as represented by the amino acid sequence as shown in SEQ ID NO: 4-6.

5. A composition according to claim 2 wherein said inhibitor of JNK2 is a siRNA molecule derived from the nucleic acid sequence in SEQ ID NO: 1-3.

6. A composition according to claim 5 wherein said JNK2 siRNA a hybrid nucleic acid molecule comprising a first part that comprises a duplex ribonucleic acid (RNA) molecule and a second part that comprises a single stranded deoxyribonucleic acid (DNA) molecule.

7. A composition according to claim 6 wherein said single stranded DNA molecule is contiguous with the sense strand of said duplex RNA molecule.

8. A composition according to claim 6 wherein said single stranded DNA molecule is contiguous with the antisense strand of said duplex RNA molecule.

9. A composition according to claim 6 wherein said single stranded DNA molecule is extended and is contiguous with both sense and antisense strands of said duplex RNA molecule.

10. A composition according to claim 6 wherein said single stranded DNA molecule comprises a 3′ terminal nucleic acid sequence wherein said sequence is adapted over at least part of its length to anneal by complementary base pairing to a part of said single stranded DNA to form a double stranded DNA structure.

11. A composition according to claim 10 wherein said single stranded DNA molecule comprises at least one copy of the sequence d (GCGAAGC).

12. A composition according to claim 6 wherein the single stranded DNA molecule is at least 7 nucleotides in length.

13. A composition according to claim 6 wherein said single stranded DNA molecule is between 10-40 nucleotide bases in length.

14. A composition according to claim 6 wherein said single stranded DNA molecule is 15-30 nucleotides in length.

15. A composition according to claim 6 wherein said duplex RNA molecule is at least 18 base pairs in length.

16. A composition according to claim 15 wherein said duplex RNA molecule is between 18 bp and 29 bp in length.

17. A composition according to claim 16 wherein said duplex RNA molecule is 21 bp in length.

18. A composition according to claim 1 wherein said inhibitor is a peptide or modified peptide.

19. A composition according to claim 1 wherein said inhibitor is a polypeptide.

20. A composition according to claim 19 wherein said polypeptide is a monoclonal antibody, or fragment thereof, that binds JNK2.

21. A composition according to claim 20 wherein said antibody fragment is a single chain antibody fragment.

22. A composition according to claim 1 wherein said chemotherapeutic agent is an anti-metabolic drug.

23. A composition according to claim 22 wherein said drug is a purine analogue.

24. A composition according to claim 22 wherein said drug is a pyrimidine analogue.

25. A composition according to claim 22 wherein said chemotherapeutic agent is selected from the group consisting of 5-fluorouracil, fluoroirinotecan and capecitabine.

26. A composition according to claim 25 wherein said chemotherapeutic agent is 5-fluorouracil.

27. A composition according to claim 25 wherein said composition includes leucovorin.

28. A method to diagnosis a disease condition that is associated with the phosphorylation of a sirtuin polypeptide comprising:
i) providing an isolated sample comprising a cell to be tested;
ii) determining the phosphorylation of at least one amino acid residue in a sirtuin polypeptide; and
iii) comparing the phosphorylation of said polypeptide in said sample to a control sample.

29. A method according to claim 28 wherein said sirtuin is SIRT1.

30. A method according to claim 29 wherein said sirtuin is represented by the amino acid of SEQ ID NO: 8.

31. A method according to claim 30 wherein said phosphorylation is at serine 27 of SEQ ID NO: 8.

32. A method according to claim 31 wherein said method includes a comparison of the phosphorylation of serine 27 with the phosphorylation of serine 47 of SEQ ID NO: 8.

33. A method according to claim 28 wherein said disease condition is a hyper-proliferative disease.

34. A method according to claim 33 wherein said disease is a viral infection caused by a pathogenic virus.

35. A method according to claim 34 wherein said disease is viral induced cancer.

36. A method according to claim 35 wherein said disease is a viral induced cancer resulting form from a human papilloma virus (HPV) infection.

37. A method according to claim 36 wherein said human papilloma virus is HPV-16 or HPV-18.

38. A method according to claim 33 wherein said hyper-proliferative disease is cancer.

39. A method according to claim 38 wherein said cell sample comprises pre-cancerous or cancerous cells.

40. A method according to claim 39 wherein said pre-cancerous or cancerous cells comprise transformed epithelial cells.

41. A method according to claim 39 wherein said pre-cancerous or cancerous cells are derived from the colon.

42. A method to diagnose a subject suffering from a condition which would benefit from a stimulation of apoptosis with a chemotherapeutic agent comprising the steps of:
i) providing an isolated sample comprising a cell to be tested;
ii) determining the phosphorylation of at least one amino acid residue in a sirtuin polypeptide;
iii) comparing the phosphorylation of said polypeptide in said sample to a control sample; and
iv) identifying a chemotherapeutic treatment regime that would benefit said subject.

43. A method according to claim 42 wherein said sirtuin is represented by the amino acid sequence of SEQ ID NO: 8.

44. A method according to claim 42 wherein said sample is additionally tested to determine the p53 genotype of said cell.

45. A method according to claim 42 wherein said at least one amino acid residue is serine 27 in SEQ ID NO: 10.

46. A method according to claim 42 wherein said method includes a comparison of the phosphorylation state of serine 27 with the phosphorylation state of serine 47 in SEQ ID NO: 10.

47-49. (canceled)

50. A method according to claim 42 wherein said cell sample comprises pre-cancerous or cancerous cells.

51. A method according to claim 50 wherein said pre-cancerous or cancerous cell comprises transformed epithelial cells.

52. A method according to claim 50 wherein said pre-cancerous or cancerous cell is derived from the colon.

53. A method according to claim 42 wherein said method further comprises the administration of a vector that includes a nucleic acid molecule that encodes a p53 polypeptide.

54. A method according to claim 53 wherein said nucleic acid molecule encoding p53 is represented by SEQ ID NO: 9.

55. A method according to claim 53 wherein said nucleic acid molecule encodes a p53 polypeptide as represented by SEQ ID NO: 10.

56. A method according to claim 42 wherein said subject is further administered an agent that inhibits the activity of the RNA-binding protein HuR.

57. A method according to claim 56 wherein said agent is an antibody or fragment thereof

58. A method according to claim 56 wherein said agent is a siRNA.

59. A method according to claim 58 wherein said siRNA is derived from SEQ ID NO: 11.

60. A method according to claim 53 wherein said nucleic acid molecule that encodes p53 is modified wherein said modification prevents or inhibits the binding of the RNA-binding protein HuR to the 5′ and/or 3′ leader sequences of p53 mRNA.

61. A method according to claim 56 wherein said subject is administered an agent that inhibits the binding of the RNA-binding protein HuR to mRNA that encodes SIRT1.

62-68. (canceled)
